(19) United States
(12) Patent Application Publication
(10) Pub. No.: US 2024/0254065 A1
(43) Pub. Date: Aug. 1, 2024

(54) METHOD FOR THE PREPARATION OF ALKENYL HALIDES

(71) Applicant: Chevron Phillips Chemical Company LP, The Woodlands, TX (US)

(72) Inventors: Qing Yang, Bartlesville, OK (US); Sierra N. Hinkle, Broken Arrow, OK (US); Mitchell D. Refvik, Bartlesville, OK (US); Graham R. Lief, Bartlesville, OK (US); Ryan N. Rose, Caney, KS (US)

(21) Appl. No.: 18/419,693

(22) Filed: Jan. 23, 2024

Related U.S. Application Data
(60) Provisional application No. 63/481,612, filed on Jan. 26, 2023.

(51) Int. Cl. C07C 17/23

(52) U.S. Cl. CPC C07C 17/23

(57) ABSTRACT

Alkenyl halides, such as an alkenyl bromide or an alkenyl chloride, are produced by a process that includes a step of forming a reaction mixture containing an alkyl dibromide or a chlorobromoalkane and an allylmagnesium bromide and/or an allylmagnesium chloride, and then producing the alkenyl halide in the reaction mixture.

### REFERENCE TO RELATED APPLICATION

This application claims the benefit of U.S. Provisional Patent Application No. 63/481,612, filed on Jan. 26, 2023, the disclosure of which is incorporated herein by reference in its entirety.

### FIELD OF THE INVENTION

The present disclosure relates generally to methods for making alkenyl halides, and more particularly, relates to synthesizing alkenyl bromides from allylmagnesium halides and alkyl dibromides and to synthesizing alkenyl chlorides from allylmagnesium halides and chlorobromoalkanes. The resulting alkenyl bromides and chlorides can be used to produce metallocene compounds with an alkenyl substituent.

### BACKGROUND OF THE INVENTION

Very few alkenyl halides are commercially available, and particularly those with four or more carbon atoms. Conventional synthesis schemes require harsh reaction conditions and the use of costly transition metal catalysts. It would therefore be beneficial to have a synthesis scheme that produces the desired alkenyl halide in high yield with mild reaction conditions and without the use of transition metal catalysts. Accordingly, it is to these ends that the present invention is generally directed.

### SUMMARY OF THE INVENTION

This summary is provided to introduce a selection of concepts in a simplified form that are further described herein. This summary is not intended to identify required or essential features of the claimed subject matter. Nor is this summary intended to be used to limit the scope of the claimed subject matter.

Processes for producing alkenyl halides are disclosed herein. For example, a process for producing an alkenyl bromide in accordance with one aspect of this invention can comprise (a) forming a reaction mixture comprising an alkyl dibromide and an allylmagnesium bromide, an allylmagnesium chloride, or a combination thereof, and (b) producing the alkenyl bromide in the reaction mixture. In this aspect, the reaction mixture can be substantially free of Li₂CuCl₄.

In another aspect of the present disclosure, a process for producing an alkenyl chloride can comprise (A) forming a reaction mixture comprising a chlorobromoalkane and an allylmagnesium bromide, an allylmagnesium chloride, or a combination thereof, at a contact temperature in a range from 15° C. to 90° C., and (B) producing the alkenyl chloride in the reaction mixture.

Both the foregoing summary and the following detailed description provide examples and are explanatory only. Accordingly, the foregoing summary and the following detailed description should not be considered to be restrictive. Further, features or variations can be provided in addition to those set forth herein. For example, certain aspects can be directed to various feature combinations and sub-combinations described in the detailed description.

### BRIEF DESCRIPTION OF THE FIGURES

FIG. 1 presents a bar chart comparing an allylmagnesium bromide diethyl ether (Et₂O) solution to an allylmagnesium chloride tetrahydrofuran (THF) solution in the synthesis of an alkenyl bromide.

FIG. 2 presents a bar chart comparing an alkyl dibromide to a chlorobromoalkane in the synthesis of alkenyl halides using an allylmagnesium chloride THF solution.

FIG. 3 presents a bar chart comparing alkyl dibromides of different chain lengths to chlorobromoalkanes of different chain lengths in the synthesis of alkenyl halides using an allylmagnesium chloride THF solution.

FIG. 4 presents a bar chart summarizing the impact of solvents on the synthesis of alkenyl bromides using an allylmagnesium chloride THF solution.

FIG. 5 presents a bar chart summarizing the impact of solvents on the synthesis of alkenyl chlorides using an allylmagnesium chloride THF solution.

FIG. 6 presents a bar chart summarizing the impact of reaction temperature on the synthesis of alkenyl bromides using an allylmagnesium bromide Et₂O solution.

FIG. 7 presents a bar chart summarizing the impact of reaction temperature on the synthesis of alkenyl chlorides using an allylmagnesium bromide Et₂O solution.

FIG. 8 presents a bar chart summarizing the impact of reaction temperature on the synthesis of alkenyl halides using an allylmagnesium chloride THF solution.

FIG. 9 presents a bar chart summarizing the impact of excess alkyl dibromide on the synthesis of alkenyl bromides using an allylmagnesium chloride THF solution.

FIG. 10 presents a bar chart summarizing the impact of reaction time on the synthesis of alkenyl bromides using an allylmagnesium chloride THF solution.

FIG. 11 presents a bar chart summarizing the impact of reaction temperature on the synthesis of alkenyl chlorides using an allylmagnesium chloride THF solution.

### DEFINITIONS

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Herein, features of the subject matter can be described such that, within particular aspects, a combination of different features can be envisioned. For each and every aspect and/or feature disclosed herein, all combinations that do not detrimentally affect the designs, compositions, processes, and/or methods described herein are contemplated with or without explicit description of the particular combination. Additionally, unless explicitly recited otherwise, any aspect and/or feature disclosed herein can be combined to describe inventive features consistent with the present disclosure.

In this disclosure, while compositions, processes/methods, and systems are described in terms of “comprising” various materials, steps, and components, the compositions, processes/methods, and systems also can “consist essentially of” or “consist of” the various materials, steps, or components, unless stated otherwise. The terms “a,” “an,” and “the” are intended to include plural alternatives, e.g., at least one, unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, transition metals for Group 3-12 elements, and halogens or halides for Group 17 elements.

For any particular compound or group disclosed herein, any name or structure presented is intended to encompass all conformational isomers, regioisomers, stereoisomers, and mixtures thereof that can arise from a particular set of substituents, unless otherwise specified. The name or structure also encompasses all enantiomers, diastereomers, and other optical isomers (if there are any), whether in enantiomeric or racemic forms, as well as mixtures of stereoisomers, as would be recognized by a skilled artisan, unless otherwise specified. For example, a general reference to hexene (or hexenes) includes all linear or branched, acyclic or cyclic, hydrocarbon compounds having six carbon atoms and 1 carbon-carbon double bond; a general reference to pentane includes n-pentane, 2-methyl-butane, and 2,2-dimethylpropane; and a general reference to a butyl group includes an n-butyl group, a sec-butyl group, an iso-butyl group, and a t-butyl group.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein. For example, the molar ratio of the alkyl dibromide (or the chlorobromoalkane) to the allylmagnesium bromide and/or allylmagnesium chloride can range from 0.8:1 to 10:1 in aspects of this invention. By a disclosure that the molar ratio can be within a range from 0.8:1 to 10:1, the intent is to recite that the molar ratio can be any molar ratio in the range and, for example, can include any range or combination of ranges from 0.8:1 to 10:1, such as from 0.8:1 to 2:1, from 1:1 to 10:1, from 1:1 to 5:1, from 1.5:1 to 10:1, from 1.5:1 to 5:1, from 1.5:1 to 4:1, from 2:1 to 6:1, or from 2:1 to 4:1, and so forth. Likewise, all other ranges disclosed herein should be interpreted in a manner similar to this example.

In general, an amount, size, formulation, parameter, range, or other quantity or characteristic is “about” or “approximate” whether or not expressly stated to be such. Whether or not modified by the term “about” or “approximately,” the claims include equivalents to the quantities or characteristics.

Although any methods and materials similar or equivalent to those described herein can be used in the practice or testing of the invention, the typical methods and materials are herein described.

All publications and patents mentioned herein are incorporated herein by reference in their entirety for the purpose of describing and disclosing, for example, the constructs and methodologies that are described in the publications and patents, which might be used in connection with the presently described invention.

### DETAILED DESCRIPTION OF THE INVENTION

Alkenyl halides are synthesized herein from allylmagnesium halides and alkyl dibromides or from allylmagnesium halides and chlorobromoalkanes in high yield, under mild reaction conditions, and without the use of transition metal catalysts.

### Alkenyl Halide Synthesis

Herein, a first process is a process for producing an alkenyl bromide, and this first process can comprise (or consist essentially of, or consist of) (a) forming a reaction mixture comprising (or consisting essentially of, or consisting of) an alkyl dibromide and an allylmagnesium bromide, an allylmagnesium chloride, or a combination thereof, and (b) producing the alkenyl bromide in the reaction mixture. The reaction mixture can be substantially free of Li₂CuCl₄. The reaction mixture also can be substantially free of other transition metal catalysts, such as Fe(acetylacetonate)₃.

The alkyl dibromide reactant utilized in the first process is not particularly limited (e.g., the alkyl dibromide can be linear or branched) and often can comprise a C₁-C₁₂ alkyl dibromide, such as a C₁-C₈ alkyl dibromide, a C₁-C₄ alkyl dibromide, a C₂-C₁₂ alkyl dibromide, a C₂-C₆ alkyl dibromide, or a C₂-C₄ alkyl dibromide, and the like. Similarly, the alkenyl bromide produced in the first process is not particularly limited (e.g., the alkenyl bromide can be linear or branched) and often can comprise a compound having the formula CH₂═CH—CH₂—(CH₂)ₙBr, wherein n is an integer from 1 to 12, from 1 to 8, or from 1 to 4 in one aspect, and from 2 to 12, from 2 to 6, or from 2 to 4 in another aspect.

A second process provided herein is a process for producing an alkenyl chloride, and this second process can comprise (or consist essentially of, or consist of) (A) forming a reaction mixture comprising (or consisting essentially of, or consisting of) a chlorobromoalkane and an allylmagnesium bromide, an allylmagnesium chloride, or a combination thereof, at a contact temperature in a range from 15° C. to 90° C., and (B) producing the alkenyl chloride in the reaction mixture.

The chlorobromoalkane reactant utilized in the second process is not particularly limited (e.g., the chlorobromoalkane can be linear or branched) and often can comprise a C₁-C₁₂ chlorobromoalkane, such as a C₁-C₈ chlorobromoalkane, a C₁-C₄ chlorobromoalkane, a C₂-C₁₂ chlorobromoalkane, a C₂-C₆ chlorobromoalkane, or a C₂-C₄ chlorobromoalkane, and the like. Similarly, the alkenyl chloride produced in the second process is not particularly limited (e.g., the alkenyl chloride can be linear or branched) and often can comprise a compound having the formula CH₂═CH—CH₂—(CH₂)ₙCl, wherein n is an integer from 1 to 12, from 1 to 8, or from 1 to 4 in one aspect, and from 2 to 12, from 2 to 6, or from 2 to 4 in another aspect.

Generally, the features of the first process and the second process (e.g., the alkenyl halide, the alkyl dibromide or chlorobromoalkane, the allylmagnesium bromide or allylmagnesium chloride, the relative amounts of the reactants, and the conditions under which the forming steps and the producing steps are conducted, among others) are independently described herein and these features can be combined in any combination to further describe the disclosed processes to produce an alkenyl halide. Moreover, additional process steps can be performed before, during, and/or after any of the steps in any of the processes disclosed herein, and can be utilized without limitation and in any combination to further describe these processes, unless stated otherwise. Further, any alkenyl halide products produced in accordance with the disclosed processes are within the scope of this disclosure and are encompassed herein.

In step (a) of the first process and step (A) of the second process, the Grignard reactant can be allylmagnesium bromide, allylmagnesium chloride, or a combination thereof. Thus, in one aspect, the reaction mixture can comprise (or consist essentially of, or consist of) the alkyl dibromide (or the chlorobromoalkane) and allylmagnesium bromide (e.g., and a solvent such as diethyl ether), while in another aspect, the reaction mixture can comprise (or consist essentially of, or consist of) the alkyl dibromide (or the chlorobromoalkane) and allylmagnesium chloride (e.g., and a solvent such as THF). Yet, in another aspect, the reaction mixture can comprise (or consist essentially of, or consist of) the alkyl dibromide (or the chlorobromoalkane) and a mixture of allylmagnesium bromide and allylmagnesium chloride (e.g., and an ether solvent).

The allylmagnesium halide can be purchased or produced prior to step (a) or (A) in the same or a nearby reactor. If purchased or produced at a distant location or a different time, the material can be stored under appropriate conditions well known to one having ordinary skill in the art, such as protected from moisture or oxygen by an inert gas. If purchased, the allylmagnesium halide can be any available concentration in an ether solvent (such as diethyl ether or THF) at concentrations up to 30 weight percent. Commonly purchased solutions are 1 or 2 molar solutions in an ether solvent.

Independently, steps (a) and (b) of the first process and steps (A) and (B) of the second process for producing the alkenyl halide can be conducted at a variety of temperatures and time periods. The time period in step (a) or step (A) is referred to as the contact time, while the time period in step (b) or step (B) is referred to as the reaction time. The contact time and the reaction time usually are different. Similarly, the temperature in step (a) or step (A) at which the alkyl dibromide (or chlorobromoalkane) and the allylmagnesium bromide and/or the allylmagnesium chloride are initially contacted to form the reaction mixture is referred to as the contact temperature, while the reaction mixture temperature in step (b) or step (B) is referred to as the reaction temperature. For instance, the contact temperature and the reaction temperature can be the same or different. As an illustrative example, in step (a), the alkyl dibromide and the allylmagnesium bromide and/or the allylmagnesium chloride can be combined initially at contact temperature T1 to form the reaction mixture and, after this initial combining, the temperature of the reaction mixture can be increased to a reaction temperature T2 to allow the (b) production of the alkenyl bromide in the reaction mixture.

The contact temperature and the reaction temperature-independently-can be a minimum temperature of 15° C., 20° C., 30° C., or 40° C.; additionally or alternatively, a maximum temperature of 90° C., 70° C., or 60° C. Generally, the contact temperature and the reaction temperature—independently—can be a temperature in a range from any minimum temperature disclosed herein to any maximum temperature disclosed herein. Therefore, suitable non-limiting contact temperature or reaction temperature ranges can include the following: from 15° C. to 90° C., from 15° C. to 70° C., from 20° C. to 90° C., from 20° C. to 70° C., from 30° C. to 90° C., from 30° C. to 70° C., or from 40° C. to 60° C. These temperature ranges also are meant to encompass circumstances where the contact temperature and/or the reaction temperature are conducted at a series of different temperatures, instead of at a single fixed temperature, wherein at least one temperature falls within the respective ranges.

Beneficially, the first process and the second process do not require a transition metal catalyst in order to achieve the surprisingly high product yields and selectivities discussed further hereinbelow. In one aspect, for instance, the reaction mixture can be substantially free of Li₂CuCl₄, i.e., the reaction mixture contains less than or equal to 1 wt. % of copper, where the weight percentage is based on the elemental weight of copper present in the reaction mixture in any form. More often, the reaction mixture contains less than or equal to 1000 ppm (by weight) of copper, less than or equal to 250 ppm of copper, less than or equal 100 ppm of copper, less than or equal to 50 ppm of copper, or less than or equal to 10 ppm of copper.

In another aspect, the reaction mixture contains less than or equal to 1 wt. %, less than or equal to 1000 ppm (by weight), less than or equal to 250 ppm, less than or equal 100 ppm, less than or equal to 50 ppm, or less than or equal to 10 ppm, of any individual transition metal, based on the elemental weight of the transition metal present in the reaction mixture in any form. The individual transition metals which are substantially absent from the reaction mixture include Group 3 through Group 12 transition metals; alternatively, Group 6 through Group 11 transition metals; or alternatively, chromium, manganese, iron (e.g., from Fe(acetylacetonate)₃), cobalt, nickel, copper, palladium, or any combination thereof.

In the first and second processes, the molar ratio of the alkyl dibromide (or the chlorobromoalkane) to the allylmagnesium bromide, the allylmagnesium chloride, or the combination thereof, is not particularly limited, and often can range from 0.8:1 to 10:1, from 0.8:1 to 2:1, from 1:1 to 10:1, or from 1:1 to 5:1. However, it can be advantageous, as demonstrated in the examples that follow, for there to be a molar excess of the alkyl dibromide in the first process and a molar excess of the chlorobromoalkane in the second process. Accordingly, suitable molar ratios of the alkyl dibromide (or the chlorobromoalkane) to the allylmagnesium bromide, the allylmagnesium chloride, or the combination thereof, can include from 1.5:1 to 10:1, from 1.5:1 to 5:1, from 1.5:1 to 4:1, from 2:1 to 6:1, or from 2:1 to 4:1, and the like.

The contact time in step (a) and step (A) of the first and second processes is not limited to any particular range. That is, the alkyl dibromide (or chlorobromoalkane) and the allylmagnesium bromide and/or the allylmagnesium chloride can be initially contacted rapidly, or over a longer period of time, before commencing the reaction and/or the production of the respective alkenyl halide in step (b) or step (B). Hence, step (a) and step (A) independently can be conducted, for example, in a time period ranging from as little as from 1-30 sec to as long as 1-6 hr. In certain aspects, the contact time can be in a range from 1 min to 2 hr., or from 5 min to 1 hr.

While the reaction time necessary to produce the desired alkenyl bromide or alkenyl chloride can vary significantly based on the reaction temperature, molar ratio of the reactants, and so forth, ordinarily the alkenyl bromide in the first process (or the alkenyl chloride in the second process) can be produced in a time period that ranges from 15 min to 10 hr. More often, the alkenyl bromide (or the alkenyl chloride) can be produced in a time period that ranges from 15 min to 5 hr., such as from 30 min to 5 hr., from 30 min to 4 hr., or from 1 hr. to 3 hr., and the like.

The respective alkenyl bromides and alkenyl chlorides produced in the first process and the second process can be formed in the presence of a solvent. Any suitable solvent can be used, although hydrocarbon solvents and ether solvents can be conveniently used. The reaction mixture in the first process and the second process, therefore, can further comprises a hydrocarbon solvent in an aspect of this invention, and illustrative and non-limiting examples of suitable hydrocarbon solvents include pentane, hexane, heptane, octane, decane, benzene, toluene, xylene, ethylbenzene, and the like. Mixtures or combinations of two or more hydrocarbon solvents can be used, if desired. In another aspect, the reaction mixture in the first process and the second process can further comprise an ether solvent, and illustrative and non-limiting examples of suitable ether solvents include dimethyl ether, diethyl ether (Et₂O), methyl ethyl ether, cyclopentyl methyl ether (CPME), methyl tert-butyl ether (MTBE), furan, dihydrofuran, tetrahydrofuran (THF), 2-methyltetrahydrofuran (2-MeTHF), 4-methyltetrahydropyran (4-MeTHP), 1,4-dioxane, and the like. Mixtures or combinations of two or more ether solvents can be used, if desired. Any relative amount of the solvent can be present in the reaction mixture, and the presence of the solvent can impact product yield and by-product formation, as shown in the examples that follow.

In an alternative aspect, the reaction mixture in the first and second processes can be substantially free of an added solvent, i.e., the reaction mixture contains less than or equal to 5 wt. % additional solvent. In this aspect, the solvent from the allylmagnesium halide solution will become part of the reaction mixture. Most commonly, this solvent is an ether solvent. More often, in this aspect, the reaction mixture can contain less than less than or equal to 1 wt. % additional solvent, or less than or equal to 0.5 wt. % additional solvent, based on the total weight of the reaction mixture.

Unexpectedly, the first process and the second process efficiently convert the respective reactants and produce the alkenyl bromide or alkenyl chloride in high yields. The molar yield of the alkenyl bromide in the reaction mixture in the first process (or the molar yield of the alkenyl chloride in the reaction mixture in the second process) can be at least 40 mol %, and more often, at least 50 mol %, at least 60 mol %, at least 70 mol %, at least 80 mol %, at least 85 mol %, or at least 90 mol %. These molar yields are based on the moles of the allylmagnesium bromide or allylmagnesium chloride reactant, or a combination thereof if more than one is utilized.

Advantageously, these high molar yields of the desired alkenyl halide often is accompanied by a relatively low amount of diene by-products. The molar amount of diene by-products in the reaction mixture generally is less than or equal to 20 mol %, and more often, less than or equal to 15 mol %, less than or equal to 10 mol %, less than or equal to 5 mol %, less than or equal to 2 mol %, or less than or equal to 1 mol %. As above, these molar yields are based on moles of the allylmagnesium bromide and/or allylmagnesium chloride reactant(s).

The first and second processes result in unexpectedly high molar selectivities of the alkenyl halide relative to the diene by-products. For instance, the molar selectivity ratio of the alkenyl bromide (or the alkenyl chloride) to the diene by-product in the reaction mixture can be at least 5:1, at least 6:1, at least 8:1, at least 10:1, at least 15:1, at least 20:1, at least 50:1, or at least 100:1; additionally or alternatively, the molar selectivity ratio can be less than or equal to 300:1, less than or equal to 250:1, less than or equal to 200:1, less than or equal to 150:1, less than or equal to 100:1, or less or equal to 50:1. Generally, the molar selectivity ratio of the alkenyl bromide (or the alkenyl chloride) to the diene by-product can be in a range from any minimum selectivity ratio disclosed herein to any maximum selectivity ratio disclosed herein. Accordingly, suitable non-limiting ranges for the molar selectivity ratio can include the following: from 5:1 to 300:1, from 5:1 to 100:1, from 6:1 to 200:1, from 6:1 to 50:1, from 8:1 to 150:1, from 10:1 to 250:1, from 10:1 to 100:1, from 15:1 to 150:1, from 20:1 to 200:1, from 50:1 to 300:1, from 100:1 to 300:1, or from 100:1 to 200:1.

Optionally, the first process and the second process can further comprise a step of quenching and, additionally or alternatively, a step of separating at least a portion (and in some cases, all) of the alkenyl bromide (or the alkenyl chloride) from the reaction mixture (after step (b) or after step (B)) to form a product mixture. Any suitable separations technique can be used, such as extraction, filtration, evaporation, distillation, and the like. Combinations of two or more of these techniques can be utilized, if desired. In an aspect, the reaction can be quenched with water, an alcohol, or mixtures thereof, either alone or in combination with a hydrocarbon solvent.

Also optionally, the first process and the second process can further comprise a step of separating at least a portion (and in some cases, all) of the alkyl dibromide (or the chlorobromoalkane) from the reaction mixture (after step (b) or after step (B)), using any suitable technique such as extraction, filtration, evaporation, distillation, and the like, or combinations thereof. After separating, the alkyl dibromide or chlorobromoalkane optionally can be recycled into the reaction mixture (in step (a) or in step (A)).

### EXAMPLES

The invention is further illustrated by the following examples, which are not to be construed in any way as imposing limitations to the scope of this invention. Various other aspects, modifications, and equivalents thereof which, after reading the description herein, can suggest themselves to one of ordinary skill in the art without departing from the spirit of the present invention or the scope of the appended claims.

### General Experimental Procedure

Synthesis: All experiments were conducted at 10 mmol scale based on AllylMgX (X═Br or Cl) under nitrogen atmosphere. AllylMgBr (1 mole/liter Et₂O solution) and allylMgCl (2 mole/liter THF solution) were purchased from Aldrich. An alkyl dihalide was charged in a flask in presence or absence of a solvent. The flask was kept in a water bath to maintain a contact temperature near room temperature (˜23° C.) and AllylMgX was added to the flask dropwise. The flask was kept in the water bath for additional 5 to 10 min after completing the addition of the AllylMgX. Then, the water bath was removed and the flask was maintained at the desired reaction temperature for a desired period of time. For the experiments with a reaction temperature higher than room temperature, the flask was heated in an oil bath to the desired reaction temperature). The reaction was quenched in the flask with water followed by addition of a known amount of toluene (toluene was used as an internal standard to quantify the products). The reaction mixture was extracted with Et₂O or pentane and then analyzed with GC/MS and GC/FID.

Product analysis: The products were confirmed by GC/MS and the corresponding standards of the analyzed compounds. The quantitative analysis of the products was conducted with GC/FID using toluene as an internal standard.

Confirmation run with product isolation: 1-bromo-3-chloropropane (2.9 mL, 29.5 mmol) was charged in a flask. The flask was kept in a water bath to maintain the contact temperature near room temperature ˜23° C.) and AllylMgCl THF solution (31 mmol, 15.5 mL of 2 mole/L in THF) was added to the flask dropwise. The flask was kept in the water bath for additional 10 min after completing the addition of the AllylMgCl solution. Then, the water bath was removed and the flask was maintained at a reaction temperature near room temperature for additional 1 hour. The reaction was quenched in the flask by water and extracted with pentane (20 mL×2). Removal of the solvent gave the product (6-chloro-1-hexene) (3.2 g, 91.4% yield) as colorless liquid with 95% purity.

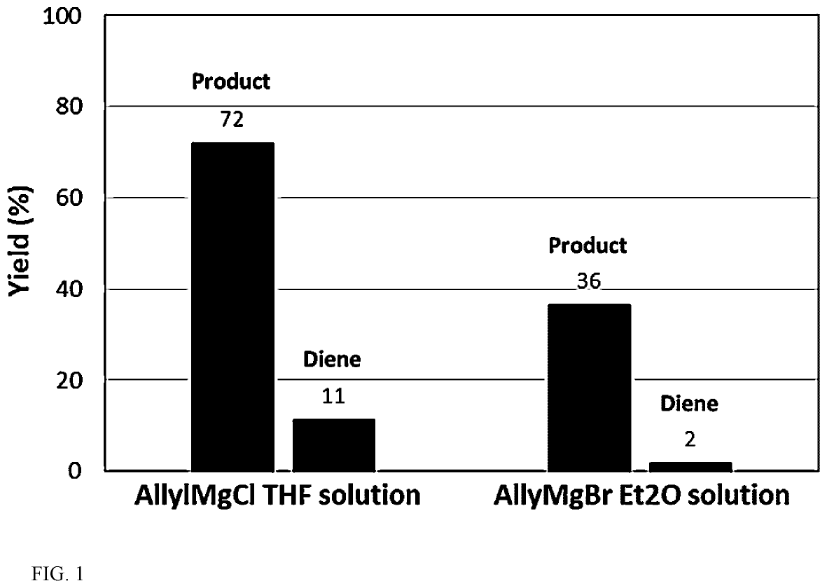

FIG. 1 demonstrates that the reaction of 1,3-dibromopropane with allylmagnesium chloride in a THF solution yielded twice as much 6-bromohex-1-ene (72 mol % based on AllylMgCl) as that resulting from the reaction of 1,3-dibromopropane with allylmagnesium bromide in Et₂O (36 mol % based on AllylMgBr), under the same reaction conditions. The use of the allylmagnesium chloride THF solution also produced significantly more diene by-product than did the allylmagnesium bromide Et₂O solution. The reaction conditions were a reactant molar ratio of 1:1 at a contact and reaction temperature of room temperature (˜23° C.) for 2 hr. in 15 mL solvent (volume ratio of diethyl ether:THF of 2:1).

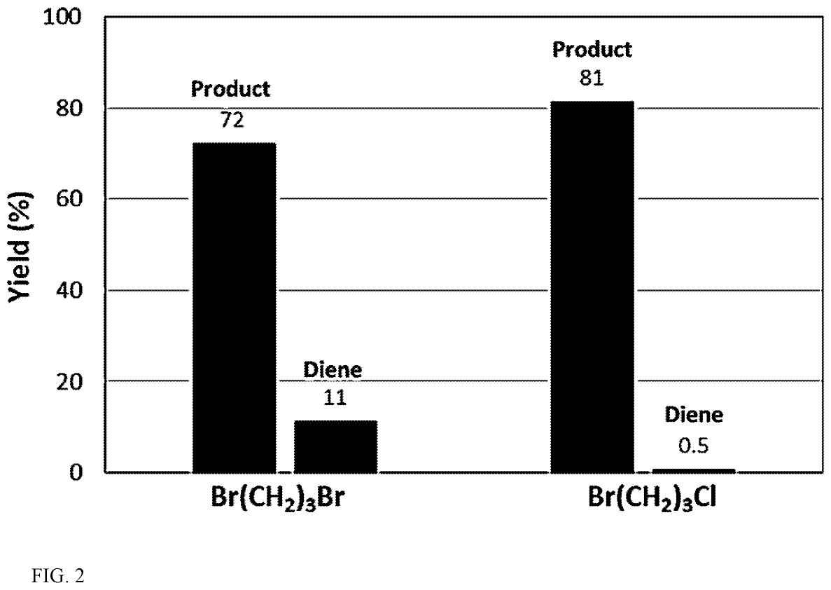

Under the same reaction conditions, FIG. 2 demonstrates that the reaction of 1-bromo-3-chloropropane with allylmagnesium chloride had a yield of 6-chlorohex-1-ene (81 mol % based on AllylMgCl) greater than the yield of 6-bromohex-1-ene from the reaction of 1,3-dibromopropane with allylmagnesium chloride (72 mol % based on AllylMgCl). The use of 1-bromo-3-chloropropane also produced significantly less diene by-product, and the molar ratio of 6-chlorohex-1-ene to diene by-product was 162:1.

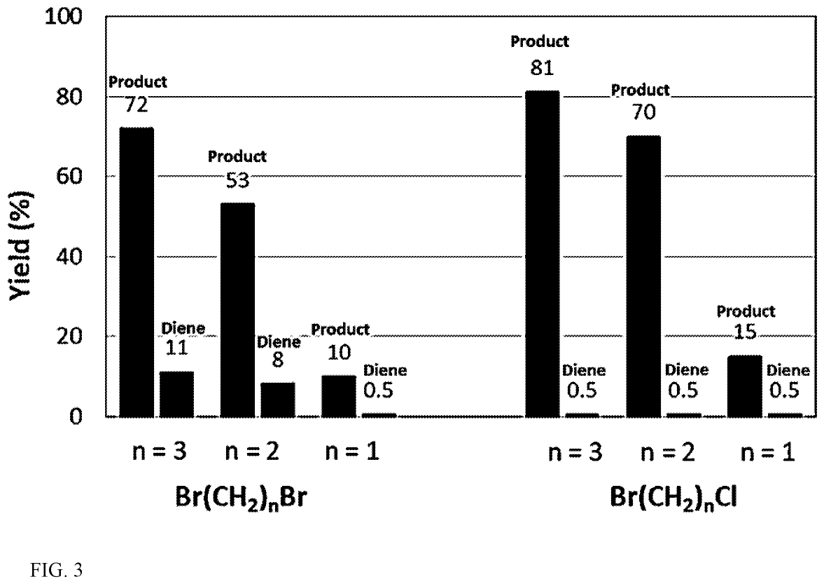

Under the same reaction conditions and using allylmagnesium chloride, FIG. 3 summarizes the impact of the chain lengths of alkyl dibromides (to produce alkenyl bromides) and the impact of the chain lengths of chlorobromoalkanes (to produce alkenyl chlorides) of different chain lengths in the synthesis of alkenyl halides. Longer dihalide chain lengths resulted in higher molar yields.

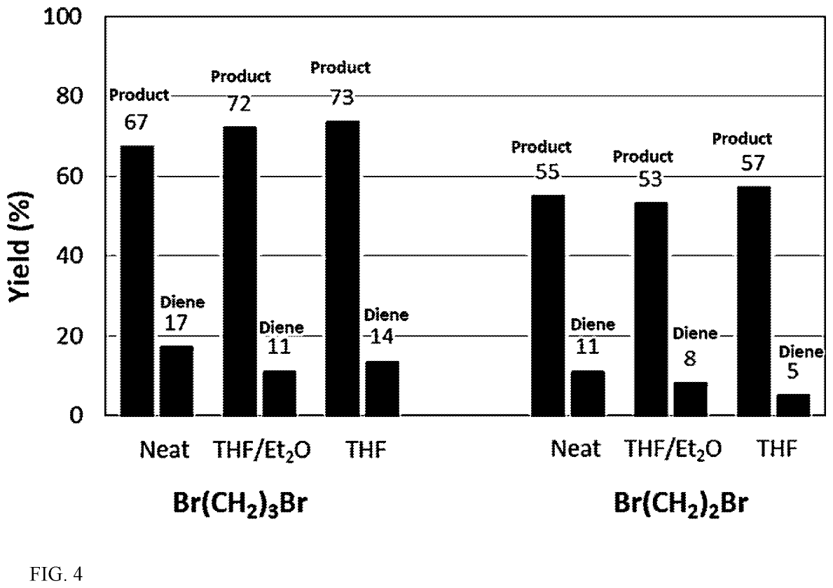

Under the same reaction conditions, FIG. 4 shows the impact of no additional solvent (neat alkyl dihalide), 15 mL of diethyl ether:THF (volume ratio 2:1, THF (5 mL) was from Grignard reagent, the alkyl dihalide was dissolved in 10 mL of Et₂O), and 15 mL of THF (THF (5 mL) was from Grignard reagent, the alkyl dihalide was dissolved in 10 mL of THF) on the yield of 6-bromohex-1-ene from the reaction of 1,3-dibromopropane with allylmagnesium chloride and on the yield of 5-bromopent-1-ene from the reaction of 1,2-dibromoethane with allylmagnesium chloride. There was no significant impact on the product yield, however, the presence of additional solvent with alkyl dihalides appeared to reduce diene by-product formation.

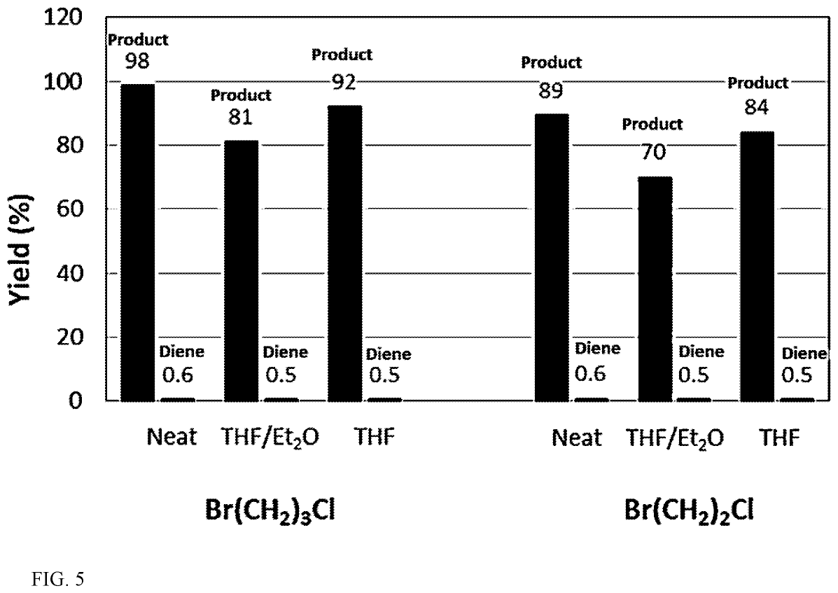

FIG. 5 is similar to FIG. 4, except that the alkyl dibromide reactant was substituted with an analogous chlorobromoalkane (and an analogous alkenyl chloride was produced). The presence of diethyl ether surprisingly reduced the product yield.

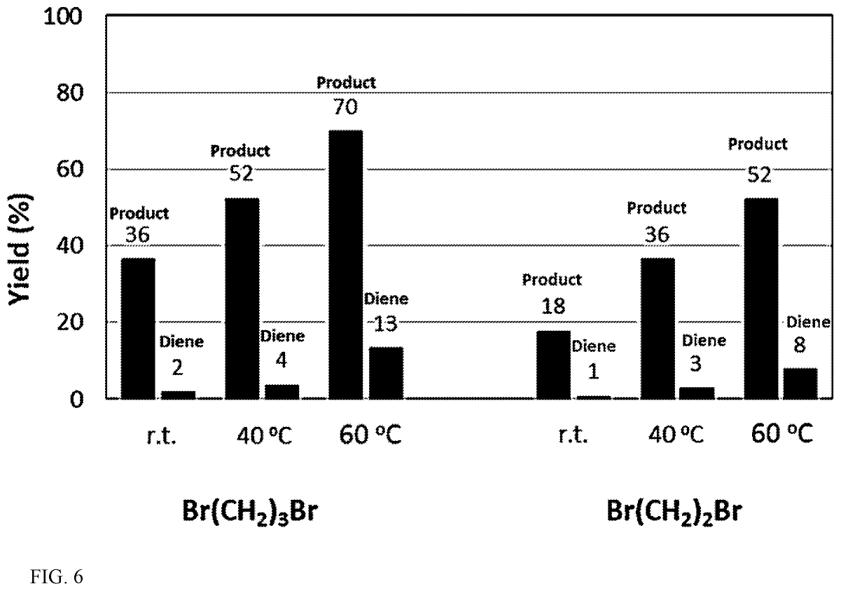

FIG. 6 summarizes the impact of reaction temperature on the synthesis of alkenyl bromides of two different chain lengths. The reactions of the allylmagnesium bromide Et₂O solution with either 1,3-dibromopropane or 1,2-dibromoethane were performed at a reactant molar ratio of 1:1 at a contact temperature of room temperature and a reaction temperature of room temperature, 40° C., or 60° C. for 2 hr. in 20 mL solvent (volume ratio of diethyl ether:THF of 1:1). Unexpectedly, the yield of the respective alkenyl bromide increased significantly with an increase in reaction temperature. The amount of diene by-product, likewise, increased with increasing reaction temperature.

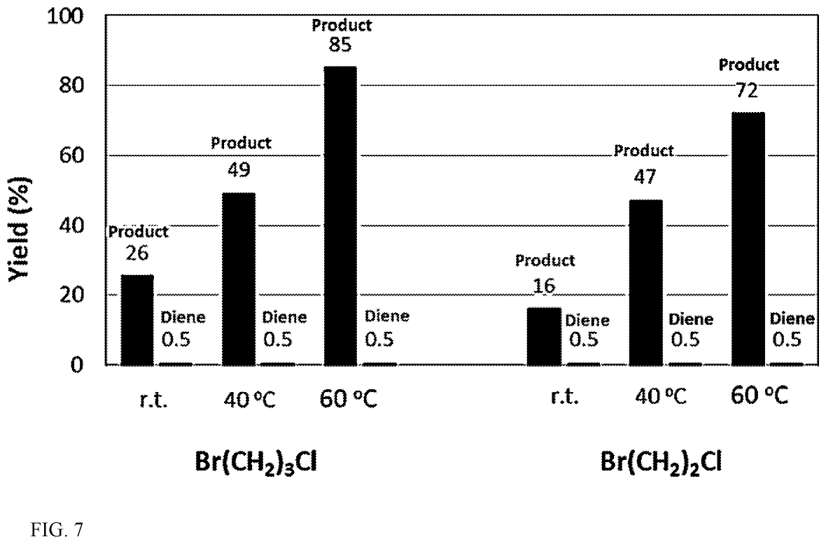

FIG. 7 is similar to FIG. 6, except that the alkyl dibromide reactant was substituted with an analogous chlorobromoalkane (and an analogous alkenyl chloride was produced). Surprisingly, the yield of the respective alkenyl chloride increased significantly with an increase in reaction temperature, while the amount of diene by-product appeared to be unaffected by reaction temperature.

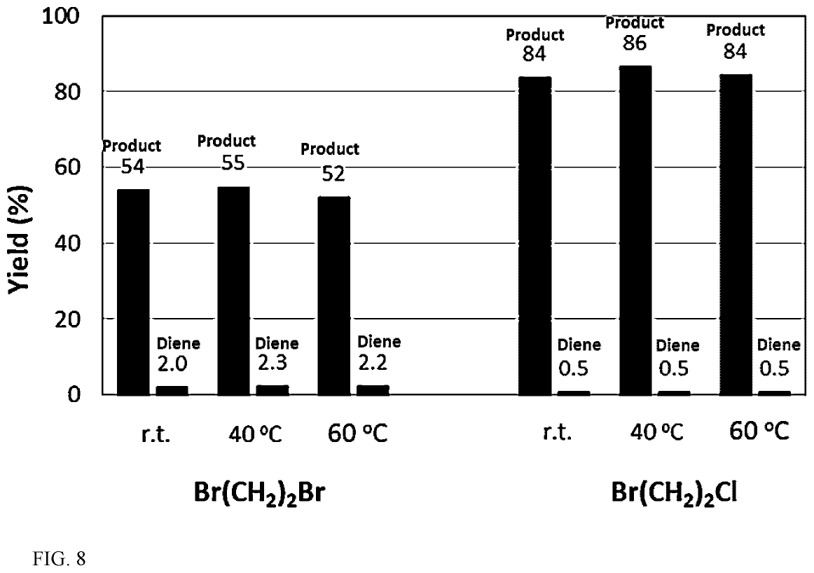

FIG. 8 is similar to FIGS. 6-7, except that the AllylMgCl THF solution was utilized instead of the AllylMgBr Et₂O solution, the reactant molar ratio of 1,2-dibromoethane:AllylMgCl was 2:1, and the reactant molar ratio of 1-bromo-2-chloroethane:AllylMgCl was 1:1. Interestingly, there was no significant impact of reaction temperature on the molar yield of the respective alkenyl halide or on the amount of diene by-product under the experimental conditions.

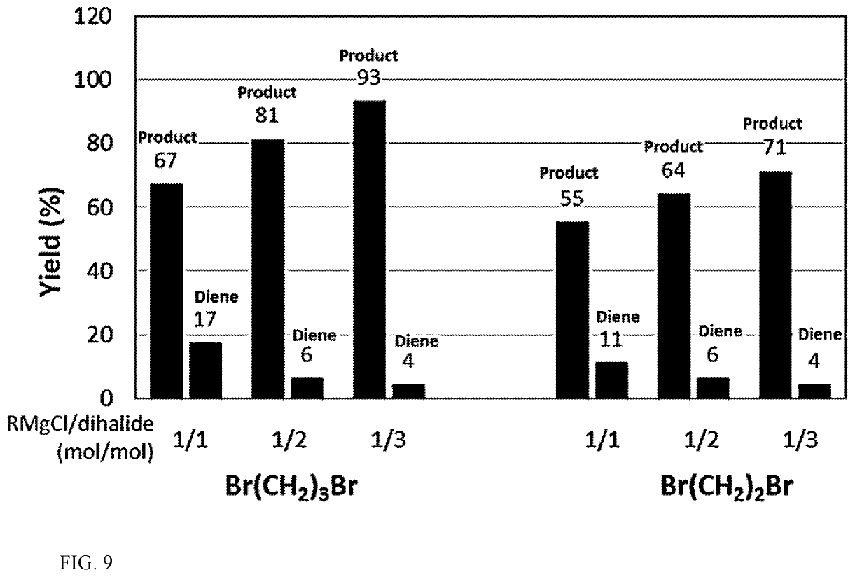

FIG. 9 summarizes the impact of excess alkyl dibromide on the synthesis of alkenyl bromides at a contact temperature and reaction temperature of room temperature for 2 hr. with no additional solvent added. As the molar ratio of dibromide:AllyMgCl increased from 1:1 to 3:1, the molar yield of the respective alkenyl bromide unexpectedly increased while the diene by-product decreased. Molar selectivity of the alkenyl bromide: diene by-product increased from 4:1-5:1 at the molar reactant ratio of 1:1 to 17:1-23:1 at the molar reactant ratio of 3:1.

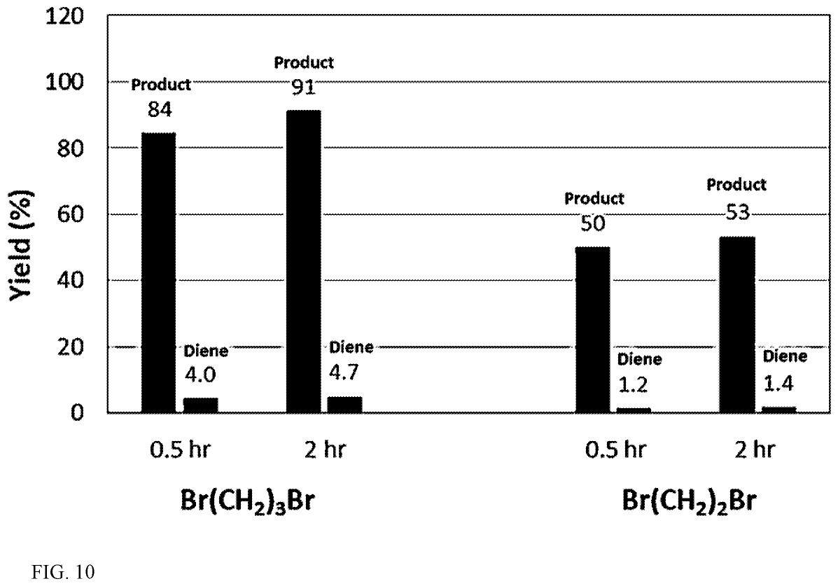

FIG. 10 summarizes the impact of reaction time on the synthesis of alkenyl bromides at a contact temperature and reaction temperature of room temperature in 15 mL THF. The reactant molar ratio of 1,2-dibromoethane:AllylMgCl was 3:1, and the reactant molar ratio of 1,3-dibromoethane:AllylMgCl was 2:1. An increase in reaction time increased the molar yield of the respective alkenyl bromide, and there was a slight increase in diene by-product under the experimental conditions.

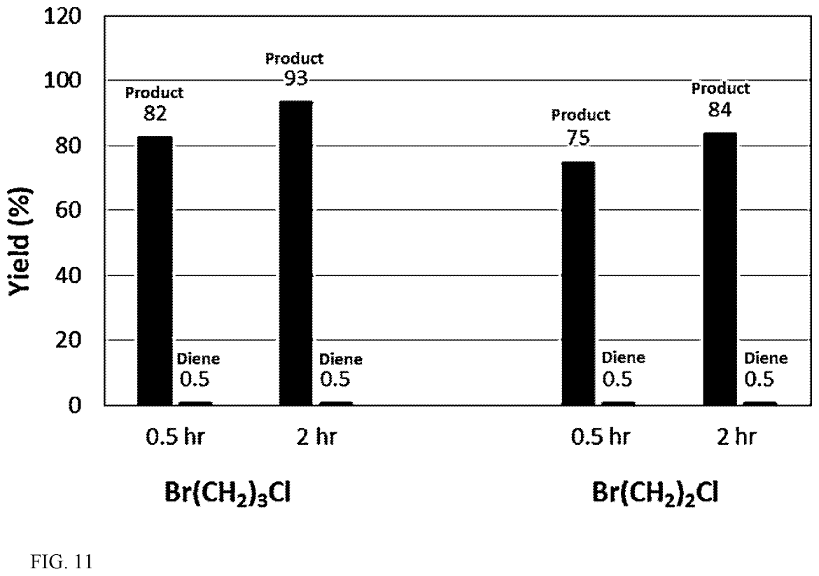

FIG. 11 is similar to FIG. 10, except that the alkyl dibromide reactant was substituted with an analogous chlorobromoalkane (and an analogous alkenyl chloride was produced), and the reactant molar ratio of chlorobromoalkane:AllylMgCl was 1:1. The yield of the respective alkenyl chloride increased significantly with an increase in reaction time, while the amount of diene by-product, surprisingly, appeared to be unaffected by reaction time.

The following general conclusions can be made from the experiments and data discussed above. The AllylMgCl THF solution is more active than the AllylMgBr Et₂O solution, and an excess of the alkyl dibromide improves product yield and reduces diene formation. Yield also is generally improved with a higher reaction temperature and a longer reaction time, and no transition metal catalyst is required to achieve high yields of the desired alkenyl halide.

The invention is described herein with reference to numerous aspects and specific examples. Many variations will suggest themselves to those skilled in the art in light of the detailed description. All such obvious variations are within the full intended scope of the appended claims. Other aspects of the invention can include, but are not limited to, the following (aspects are described as “comprising” but, alternatively, can “consist essentially of” or “consist of”):

Aspect 1. A process for producing an alkenyl bromide, the process comprising (a) forming a reaction mixture comprising (or consisting essentially of, or consisting of) an alkyl dibromide and an allylmagnesium bromide, an allylmagnesium chloride, or a combination thereof, and (b) producing the alkenyl bromide in the reaction mixture, wherein the reaction mixture is substantially free of Li₂CuCl₄.

Aspect 2. The process defined in aspect 1, wherein the alkyl dibromide comprises a C₁-C₁₂ alkyl dibromide, a C₁-C₈ alkyl dibromide, a C₁-C₄ alkyl dibromide, a C₂-C₁₂ alkyl dibromide, a C₂-C₆ alkyl dibromide, or a C₂-C₄ alkyl dibromide.

Aspect 3. The process defined in aspect 1 or 2, wherein the alkenyl bromide comprises a compound having the formula CH₂═CH—CH₂—(CH₂)ₙBr, wherein n is an integer from 1 to 12, from 1 to 8, from 1 to 4, from 2 to 12, from 2 to 6, or from 2 to 4.

Aspect 4. A process for producing an alkenyl chloride, the process comprising (A) forming a reaction mixture comprising (or consisting essentially of, or consisting of) a chlorobromoalkane and an allylmagnesium bromide, an allylmagnesium chloride, or a combination thereof, at a contact temperature in a range from 15 to 90° C., and (B) producing the alkenyl chloride in the reaction mixture, for example, at a reaction temperature in a range from 15 to 90° C.

Aspect 5. The process defined in aspect 4, wherein the chlorobromoalkane comprises a C₁-C₁₂ chlorobromoalkane, a C₁-C₈ chlorobromoalkane, a C₁-C₄ chlorobromoalkane, a C₂-C₁₂ chlorobromoalkane, a C₂-C₆ chlorobromoalkane, or a C₂-C₄ chlorobromoalkane.

Aspect 6. The process defined in aspect 4 or 5, wherein the alkenyl chloride comprises a compound having the formula CH₂50 CH—CH₂—(CH₂)ₙCl, wherein n is an integer from 1 to 12, from 1 to 8, from 1 to 4, from 2 to 12, from 2 to 6, or from 2 to 4.

Aspect 7. The process defined in any one of aspects 1-6, wherein the reaction mixture comprises (or consists essentially of, or consists of) the alkyl dibromide (or the chlorobromoalkane), the allylmagnesium bromide, and a solvent (e.g., Et₂O).

Aspect 8. The process defined in any one of aspects 1-6, wherein the reaction mixture comprises (or consists essentially of, or consists of) the alkyl dibromide (or the chlorobromoalkane), the allylmagnesium chloride, and a solvent (e.g., THF).

Aspect 9. The process defined in any one of aspects 1-8, wherein the reaction mixture in step (a) or step (A) is formed at a contact temperature in a range from 15 to 90° C. or a contact temperature in any range disclosed herein, e.g., from 15 to 70° C., from 20 to 90° C., from 20 to 70° C., from 30 to 90° C., from 30 to 70° C., or from 40 to 60° C.

Aspect 10. The process defined in any one of aspects 1-9, wherein the alkenyl bromide in step (b) or the alkenyl chloride in step (B) is produced at a reaction temperature in a range from 15 to 90° C.or a reaction temperature in any range disclosed herein, e.g., from 15 to 70° C., from 20 to 90° C., from 20 to 70° C., from 30 to 90° C., from 30 to 70° C., or from 40 to 60° C.

Aspect 11. The process defined in any one of aspects 1-10, wherein the reaction mixture is substantially free of Li₂CuCl₄, i.e., the reaction mixture contains less than or equal to 1 wt. % of copper (elemental basis).

Aspect 12. The process defined in any one of aspects 1-10, wherein the reaction mixture contains less than or equal to 1000 ppm (by weight), less than or equal to 250 ppm, less than or equal 100 ppm, less than or equal to 50 ppm, or less than or equal to 10 ppm, of copper (elemental basis).

Aspect 13. The process defined in any one of aspects 1-12, wherein the reaction mixture contains less than or equal to 1 wt. %, less than or equal to 1000 ppm (by weight), less than or equal to 250 ppm, less than or equal 100 ppm, less than or equal to 50 ppm, or less than or equal to 10 ppm, of a transition metal (elemental basis), e.g., nickel, iron, palladium, etc.

Aspect 14. The process defined in any one of aspects 1-13, wherein a molar ratio of the alkyl dibromide (or the chlorobromoalkane) to the allylmagnesium bromide, the allylmagnesium chloride, or the combination thereof, is in any range disclosed herein, e.g., from 0.8:1 to 10:1, from 0.8:1 to 2:1, from 1:1 to 10:1, from 1:1 to 5:1, from 1.5:1 to 10:1, from 1.5:1 to 5:1, from 1.5:1 to 4:1, from 2:1 to 6:1, or from 2:1 to 4:1.

Aspect 15. The process defined in any one of aspects 1-14, wherein the alkenyl bromide (or the alkenyl chloride) is produced in a time period in a range from 15 min to 10 hr., from 15 min to 5 hr., from 30 min to 5 hr., from 30 min to 4 hr., or from 1 hr. to 3 hr.

Aspect 16. The process defined in any one of aspects 1-15, wherein a molar yield of the alkenyl bromide (or the alkenyl chloride) in the reaction mixture, based on the allylmagnesium bromide, the allylmagnesium chloride, or the combination thereof, is at least 40%, at least 50%, at least 60%, at least 70%, at least 80%, at least 85%, or at least 90%.

Aspect 17. The process defined in any one of aspects 1-16, wherein a molar amount of diene by-product in the reaction mixture, based on the allylmagnesium bromide, the allylmagnesium chloride, or the combination thereof, is less than or equal to 20%, less than or equal to 15%, less than or equal to 10%, less than or equal to 5%, less than or equal to 2%, or less than or equal to 1%.

Aspect 18. The process defined in any one of aspects 1-17, wherein a molar selectivity ratio of the alkenyl bromide (or the alkenyl chloride) to a diene by-product in the reaction mixture is at least 5:1, at least 6:1, at least 8:1, at least 10:1, at least 15:1, at least 20:1, at least 50:1, or at least 100:1; and less than or equal to 300:1, less than or equal to 250:1, less than or equal to 200:1, less than or equal to 150:1, less than or equal to 100:1, or less or equal to 50:1; or any suitable range from any minimum value to any maximum value.

Aspect 19. The process defined in any one of aspects 1-18, wherein the reaction mixture is substantially free of an additional solvent except from the Grignard reagents (i.e., the reaction mixture contains less than or equal to 5 wt. % additional solvent except from the Grignard reagents), or the reaction mixture contains less than or equal to 1 wt. % additional solvent except from the Grignard reagents, or less than or equal to 0.5 wt. %

additional solvent except from the Grignard reagents, based on the total weight of the reaction mixture.

Aspect 20. The process defined in any one of aspects 1-18, wherein the reaction mixture further comprises any suitable solvent, e.g., an ether solvent selected from dimethyl ether, diethyl ether, methyl ethyl ether, furan, dihydrofuran, tetrahydrofuran (THF), or any combination thereof; and/or a hydrocarbon solvent selected from pentane, hexane, heptane, octane, decane, benzene, toluene, xylene, ethylbenzene, or any combination thereof.

Aspect 21. The process defined in any one of aspects 1-20, further comprising a step of separating at least a portion (and in some cases, all) of the alkenyl bromide (or the alkenyl chloride) from the reaction mixture (after step (b) or after step (B)) to form a product mixture, using any suitable technique or any technique disclosed herein, e.g., extraction, filtration, evaporation, distillation, etc., or any combination thereof.

Aspect 22. The process defined in any one of aspects 1-21, further comprising a step of a separating at least a portion (and in some cases, all) of the alkyl dibromide (or the chlorobromoalkane) from the reaction mixture (after step (b) or after step (B)), using any suitable technique or any technique disclosed herein, e.g., extraction, filtration, evaporation, distillation, etc., or any combination thereof, and optionally recycling into the reaction mixture (in step (a) or in step (A)).

What is claimed is:

1. A process for producing an alkenyl bromide, the process comprising:
(a) forming a reaction mixture comprising an alkyl dibromide and an allylmagnesium bromide, an allylmagnesium chloride, or a combination thereof; and
(b) producing the alkenyl bromide in the reaction mixture;
wherein the reaction mixture is substantially free of Li₂CuCl₄.

2. The process of claim 1, wherein the alkyl dibromide comprises a C₁-C₁₂ alkyl dibromide.

3. The process of claim 1, wherein the alkenyl bromide comprises a compound having the formula CH₂═CH—CH₂—(CH₂)ₙBr, and wherein n is an integer from 1 to 12.

4. The process of claim 1, wherein the reaction mixture comprises the alkyl dibromide and the allylmagnesium bromide.

5. The process of claim 1, wherein the reaction mixture comprises the alkyl dibromide and the allylmagnesium chloride.

6. The process of claim 1, wherein:
the reaction mixture in step (a) is formed at a contact temperature in a range from 15° C. to 90° C.; and
the alkenyl bromide in step (b) is produced at a reaction temperature in a range from 15° C. to 90° C.

7. The process of claim 1, wherein the alkenyl bromide in step (b) is produced at a reaction temperature in a range from 30° C. to 70° C.

8. The process of claim 1, wherein a molar ratio of the alkyl dibromide to the allylmagnesium bromide, the allylmagnesium chloride, or the combination thereof, is in a range from 1.5:1 to 10:1.

9. The process of claim 1, wherein:
a molar yield of the alkenyl bromide in the reaction mixture, based on the allylmagnesium bromide, the allylmagnesium chloride, or the combination thereof, is at least 50%; and
a molar amount of diene by-product in the reaction mixture, based on the allylmagnesium bromide, the allylmagnesium chloride, or the combination thereof, is less than or equal to 15%.

10. The process of claim 1, wherein a molar selectivity ratio of the alkenyl bromide to a diene by-product in the reaction mixture is from 5:1 to 250:1.

11. The process of claim 1, wherein the reaction mixture further comprises an ether solvent and/or a hydrocarbon solvent.

12. The process of claim 1, wherein no additional solvent is used, and solvent present in the reaction mixture is from a solution of the allylmagnesium bromide, the allylmagnesium chloride, or the combination thereof.

13. The process of claim 1, wherein the process further comprises a step of separating at least a portion of the alkenyl bromide from the reaction mixture after step (b) to form a product mixture.

14. The process of claim 1, wherein the process further comprises a step of separating at least a portion of the alkyl dibromide from the reaction mixture after step (b), and recycling into the reaction mixture in step (a).

15. A process for producing an alkenyl chloride, the process comprising:
(A) forming a reaction mixture comprising a chlorobromoalkane and an allylmagnesium bromide, an allylmagnesium chloride, or a combination thereof, at a contact temperature in a range from 15° C. to 90° C.; and
(B) producing the alkenyl chloride in the reaction mixture.

16. The process of claim 15, wherein:
the alkenyl chloride in step (B) is produced at a reaction temperature in a range from 30° C. to 70° C.; and/or
the chlorobromoalkane comprises a C₁-C₁₂ chlorobromoalkane.

17. The process of claim 15, wherein the reaction mixture comprises the chlorobromoalkane and the allylmagnesium bromide.

18. The process of claim 15, wherein the reaction mixture comprises the chlorobromoalkane and the allylmagnesium chloride.

19. The process of claim 15, wherein:
the alkenyl chloride comprises a compound having the formula CH₂═CH—CH₂—(CH₂)ₙCl, and wherein n is an integer from 1 to 12; or
a molar ratio of the chlorobromoalkane to the allylmagnesium bromide, the allylmagnesium chloride, or the combination thereof, is in a range from 1:1 to 10:1; or
a molar selectivity ratio of the alkenyl chloride to a diene by-product in the reaction mixture is from 5:1 to 250:1; or
any combination thereof.

20. The process of claim 15, wherein:
a molar yield of the alkenyl chloride in the reaction mixture, based on the allylmagnesium bromide, the allylmagnesium chloride, or the combination thereof, is at least 50%; and
a molar amount of diene by-product in the reaction mixture, based on the allylmagnesium bromide, the allylmagnesium chloride, or the combination thereof, is less than or equal to 15%.
